(19) United States
(12) Patent Application Publication
(10) Pub. No.: US 2011/0112366 A1
(43) Pub. Date: May 12, 2011

(54) Laparoscopic instrument

(76) Inventors: Nauman Basit, Lahore (PK); Aqeel Abbas, Sialkot (PK)

(21) Appl. No.: 12/614,410

(22) Filed: Nov. 7, 2009

(51) Int. Cl. A61B 1/045

(52) U.S. Cl. 600/131

(57) ABSTRACT

An improved laparoscopic instrument with an ergonomic handle comprising a large fixed ring for three fingers and a small moveable thumb ring. The invention enables a surgeon to use all controls on the instrument, including opening and closing of jaws, rotating and ratcheting them with one hand. The invention introduces a unique placement of ratchet mechanism in the large fixed ring where its use is possible with a very intuitive movement of middle and ring finger. The circular rotating knob is designed to be large in size, with large lateral projections and placed at a distance to be easily actuated. Its smooth rotation is ensured through a frictionless rotary bearing that also provides a solid fastening with the elongate tube. The high frequency (HF) connector is designed to ensure a definite and safe contact with the elongate conduit.

### FIELD OF INVENTION

Laparoscopic instruments are used in laparoscopic surgery, also called minimally invasive surgery (MIS), band aid surgery or keyhole surgery. It is a modern surgical technique in which operations in the abdominal or pelvic cavity are performed through 3 to 4 small incisions (usually 0.5-1.5 cm) as compared to larger incisions needed in traditional surgical procedures. Various kinds of instruments like grasping forceps, dissecting forceps and scissors along with some visualizing device are inserted through these holes using trocar and surgery is performed.

### BACKGROUND

Laparoscopic instruments comprise an operating handle, an insulated elongate conduit (6) for passing the instrument through a trocar into the abdominal cavity, and small working components (8) such as grasping forceps, dissecting forceps and scissors jaws pivoted to the distal operating end of the elongate conduit (6). The elongate conduit (6) encases a rigid linkage connecting the operating end to the handle in such a way that manipulation of the handle causes the working components (8) at the operating end to open and close. In operation it is usually desired to orient the working end at appropriate angles as well, so a rotating mechanism is employed at the handle to rotate the elongate conduit 6), up to 360 degrees, along the longitudinal axis of the conduit, in both clockwise and counterclockwise directions. In addition to these basic functions, a critical feature required by the surgeon is to be able to clamp the object at the operating end, such as a gall bladder in Cholysystectomy. A ratchet mechanism has to be incorporated at the handle so that by manipulating the mechanism, the working components (8) can operate in a one-way clamping direction only and grasp the object.

In many conventional instruments, the handle comprises two holes for insertion of middle finger in one ring and thumb in the other ring. The sizes of these rings are often small and not optimized for all types of hand sizes. This method in which the whole instrument is supported by only a finger and thumb and in which case, the hand and wrists make a very awkward and unnatural angle with respect to the angle of use is often very cumbersome to the surgeon and extended use of instrument in this position causes severe fatigue and hand pain. This results in painful situations during extended surgeries.

In conventional laparoscopic instruments, controls for the respective mechanisms are placed at such locations where a definite effort has to be made to reach and operate them by one hand, in most cases requiring the support of the other hand for rotating the conduit or engaging the ratchet mechanism, which results in difficulty for the surgical procedures.

Repeated long term operation causes fatigue to the hand and wrist as well. There is a definite need for an ergonomic laparoscopic instrument that enables the surgeon to use only one hand in a comfortable position to open, close, rotate and ratchet the operating end at any given position while applying the desired clamping force.

### SUMMARY

The present invention is directed to an improved laparoscopic instrument and comprises an ergonomic handle design which enables the surgeon to use only one hand to orient instrument working components (8) into desired operating angle, ratcheting their grasping action while applying the desired clamping force and for actuating them as required for a surgical procedure. The improved instrument is made of biocompatible engineering thermoplastic and medical grade stainless steel, with due consideration to being aesthetically appealing, comfortable, intuitive and dexterous in use, functionally superior and ergonomically advantageous.

A plastic instrument body can be used and the body comprises a grip handle that extends downwardly from a zeppelin shaped oval belly (1). The oval belly (1) may also be referred to as a housing. The handle comprises a large fixed ring (4) to accommodate middle, ring and little finger and a small movable thumb ring (5) for thumb insertion. The index finger sits nicely in a trigger groove (15) under the zeppelin shaped oval belly (1) and above the large three finger ring. A circular wheel (3) with four large radial levers (28) at the circumference is placed at the front of the zeppelin shaped oval belly (1) for rotating the elongate conduit (6) and in effect, the working components (8). The wheel (3) may also be referred to as a knob. As a part of the ratchet mechanism, an external locking slider (9) is placed along the inner side of the large fixed ring (4). It is positioned such that it is held between the middle finger on top and ring finger below. Using these fingers, the external locking slider (9) is moved up and down to activate or deactivate the ratchet mechanism. This arrangement ensures placement of all fingers on the instrument that brings all controls within a comfortable range for precise manipulation.

The elongate conduit (6) exits the zeppelin shaped oval belly (1) through the circular wheel (3) and extends distally to connect with the working components (8) at the operating end. The working components (8) assembly comprises a small tube which is split at the front to encapsulate a pair of working components (8) assembled at the pivot to the split tube (7). The elongate conduit (6) is insulated to protect the outer surface from high frequency current which passes through the conduit.

There is also a high frequency (HF) cable connector (2) that protrudes form the top of zeppelin shaped oval belly (1) which is internally connected to the elongate conduit (6). The HF cable connector (2) provides a means for connection with an HF cable.

As a result, the invention enables a surgeon to perform complex surgical procedures by manipulating all the control mechanisms with one hand in a comfortable and precise manner.

### BRIEF DESCRIPTION OF THE DRAWINGS

One or more embodiments of the invention are shown in the form of drawings and illustration to show the internal mechanisms. The details of these drawings are as follows:

FIG. 1. is a diagram of a laparoscopic instrument.

FIG. 2. is a right side elevation of the handle in which portions of the instrument frame have been cut away to show the internal details.

FIG. 3. is a partial view of the instrument that shows the ratchet mechanism in a cut away portion of the instrument frame to show various components.

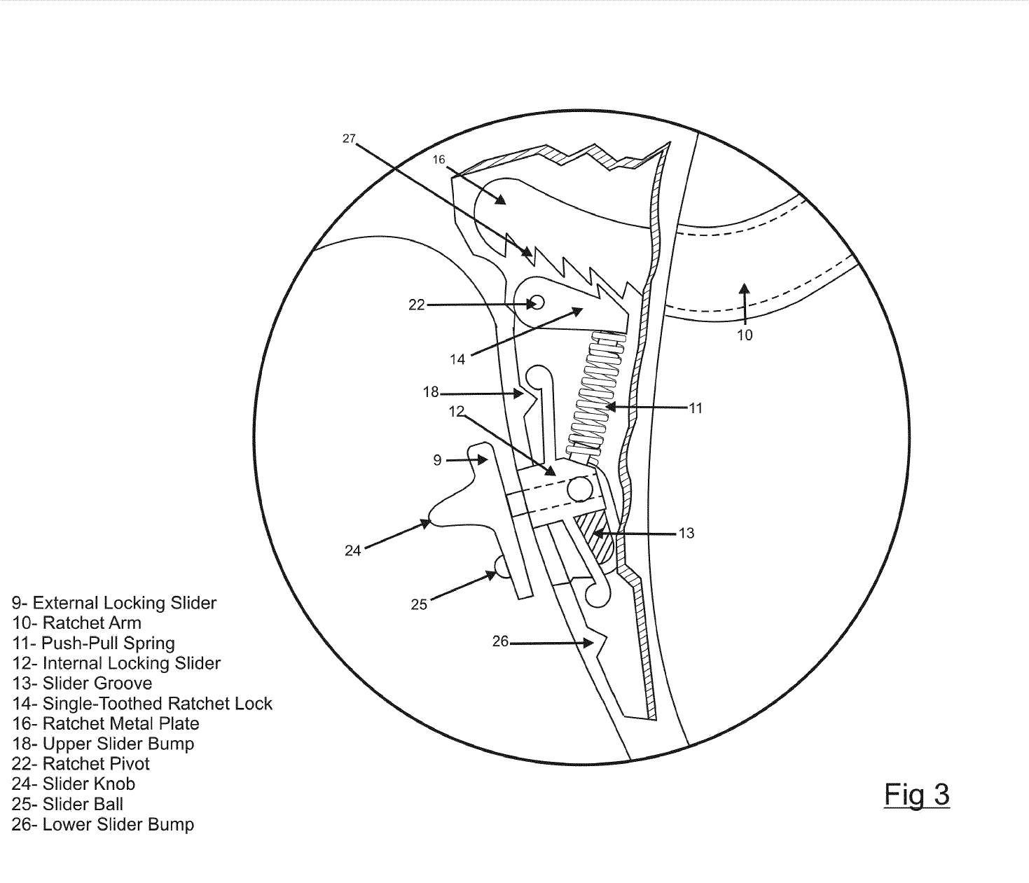

FIG. 4. is a diagram of an HF connector assembly.

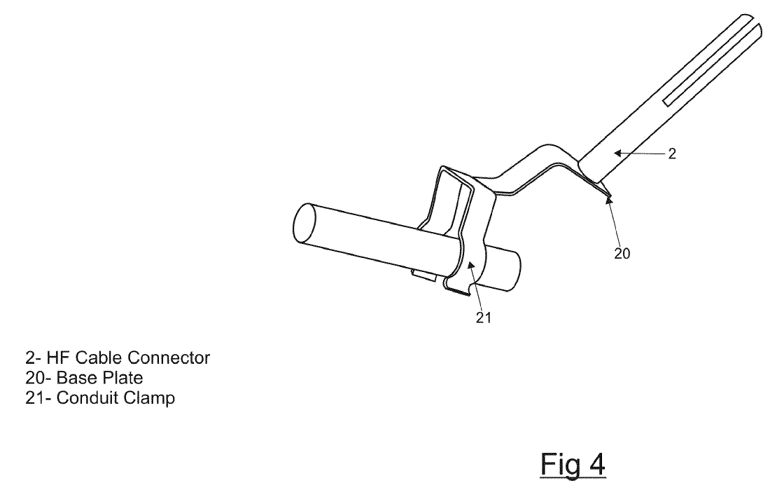

FIG. 5. shows a rotary bearing and its method of fastening to an elongate conduit.

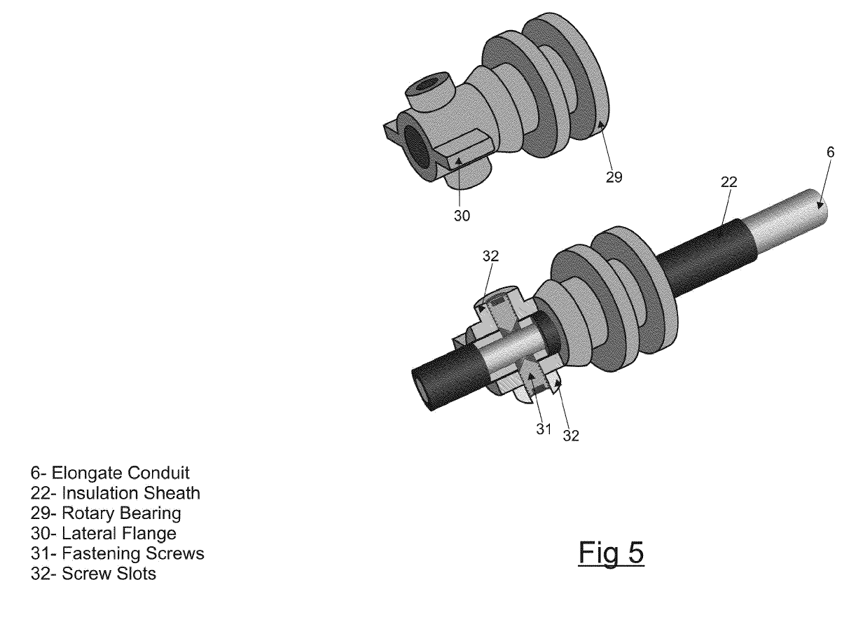

### DETAILED DESCRIPTION

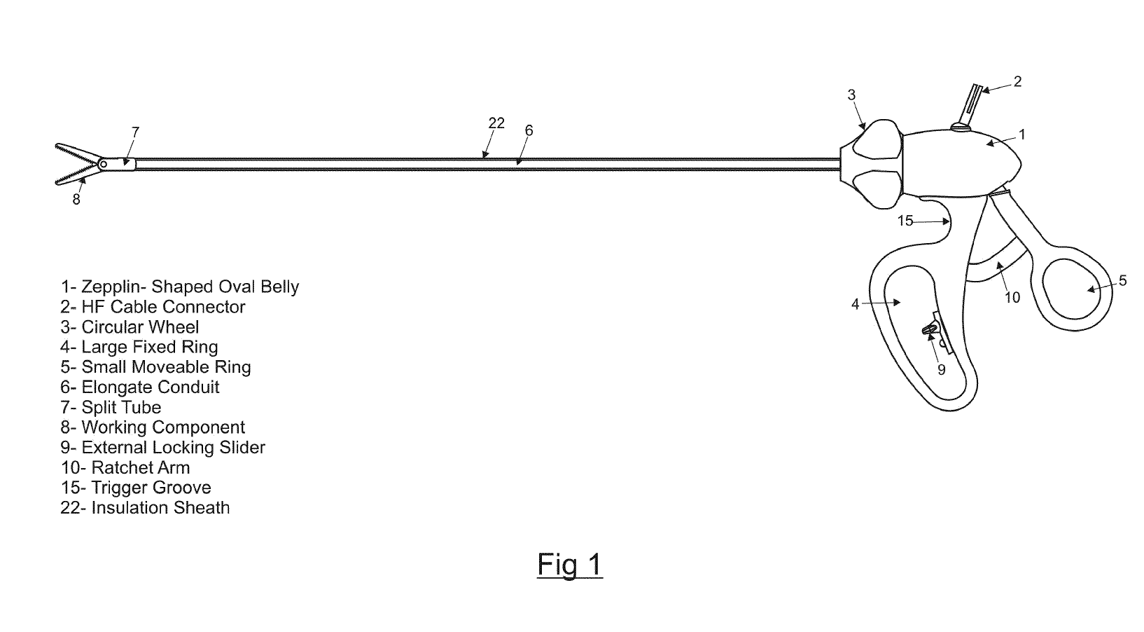

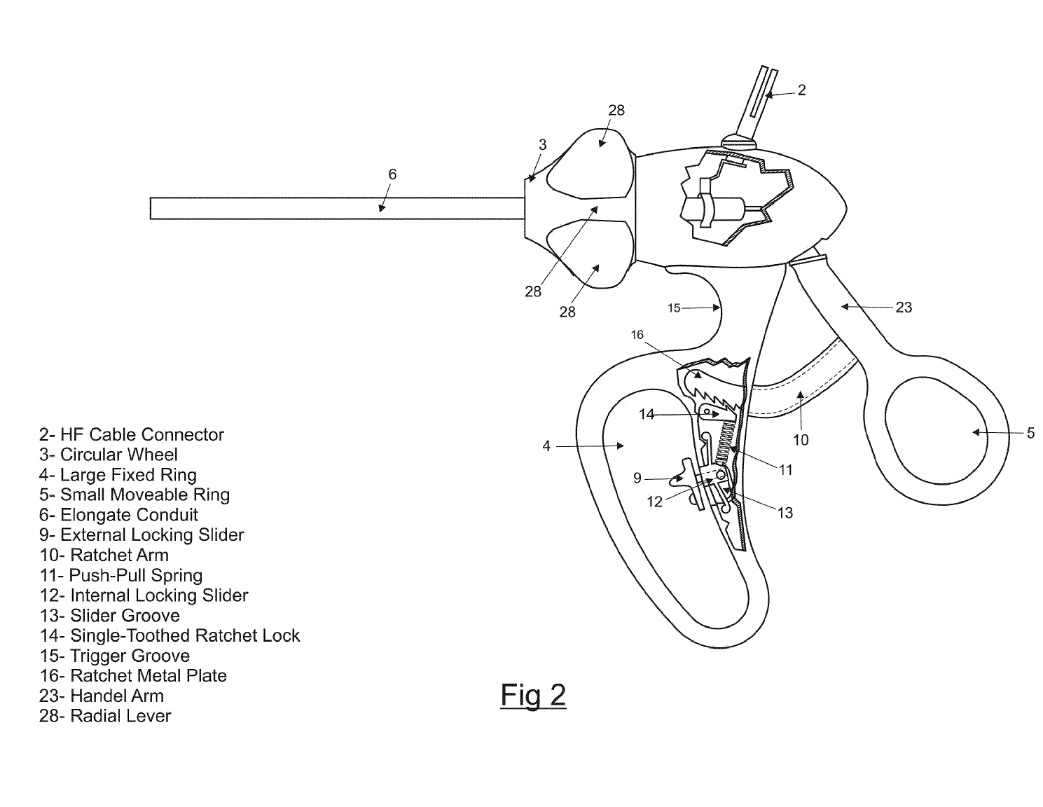

The overall design, as depicted in FIG. 1. and FIG. 2, gives a very unique, original and ergonomic look. The zeppelin-shaped oval belly (1) of instrument is unique and provides a distinct and distinguished profile. This zeppelin shaped oval belly (1) of instrument provides a central platform, from which the handle extends downward. The HF cable connector (2) protrudes from the top side and the circular wheel (3) is attached in the front part.

The ergonomic handle comprises two rings, one large fixed ring (4) and one small moveable ring (5). The large fixed ring (4) is part of the zeppelin-shaped oval belly (1) and connects with it at the top. The small moveable ring (5) is connected with a handle arm (23) and enters the zeppelin shaped oval belly (1) posterior, through a pivotal arrangement.

The HF assembly comprises an external HF cable connector (2), a base plate (20) and a conduit clamp (21). The design of this HF assembly is such that the clamping part makes a definite sliding contact with the elongate tube (6).

The zeppelin shaped oval belly (1) also provides a large room for housing the inside working mechanism of the instrument. The whole idea makes the design very stable.

When the instrument is held in hand, with fingers placed in the large fixed ring (4), the index finger placed in the trigger groove (15) and the thumb placed in the small moveable ring (5), the overall position of the fingers, hand and wrist in this engaged position remains natural and undistorted. This also ensures a very ergonomic way of holding the entire instrument in hand. The angle of grip is comparatively more in line with the angle of use and hence, it is possible to use the instrument over an extended period of time without pain and fatigue.

The large fixed ring (4) is so designed as to be large and spacious enough to easily accommodate the middle, the ring and little finger, which can sit very ergonomically and comfortably in the ring. The size of the inner diameter of the large fixed ring (4) is optimized to accommodate hands of all size and shapes.

Through this arrangement all fingers and thumb are secured in the handle giving a comfortable and ergonomic feeling to the surgeon than when one or two fingers are dangling outside the ring and are unsupported in many conventional instruments.

Immediately above the large fixed ring (4), there is a large trigger groove (15) where the index finger can comfortably rest and intuitively operate during the surgical operations. From this position, the ring finger can exert a corresponding opposing force required for the clamping action, actuated through the thumb.

The small moveable ring (5) is also optimized so that it allows for a very comfortable placement for thumbs of all types and sizes. It is neither too big to be a problem for smaller hands, nor too small to be a problem for larger hands.

The invention introduces a unique ratchet mechanism that comprises an external locking slider (9), an internal locking slider (12) a ratchet arm (10) and a single-toothed ratchet lock (14), which is secured in its position and operates through a push-pull spring (11), which pulls it down or pushes it up by the corresponding movement of the internal slider lock (12) through a ratchet pivot (22).

The external slider lock (9) comprises a projected slider knob (24), in its middle part and a small projected ball (25) at the base. It is strategically placed near the inside of the large fixed ring (4) between middle and ring finger. It is connected internally with the internal slider lock (12). When the slider knob (24) is moved up, the internal slider lock (12) also moves up until it pushes over the upper slider bump (18) and gets locked at this position. This upward movement pushes the push-pull spring (11) upwards which in turn pushes the single-toothed ratchet lock (14), which engages with the downward projected ratchet teeth (27) on the ratchet arm (10). This way the ratchet is activated for the movement of small moveable ring (5). In this manner, any object or tissue clamped in the working components (8) gets permanently grasped, which enables the surgeon to perform other surgical procedures without losing this grip.

To deactivate the ratchet (10), the surgeon pushes the slider knob (24) downwards using his middle finger. The corresponding downward movement of the internal slider lock (12) pulls the push-pull spring (11) downwards; the single-toothed ratchet lock (14) disengages the ratchet teeth which become free to move to and fro by the action of moveable small ring (5). When the sliders are moved further down, they are pulled over the lower slider bump (26) and get locked in this position. This allows the surgeon to use the instrument without ratcheting.

The ratchet arm (10) comprises a metal plate (16) that has pointed teeth at the distal half of the bottom side and is covered on top and bottom with plastic covers for user safety.

The circular wheel (3), which rotates the shaft and the working components (8) to 360 degrees is bigger in size. Its laterally projected radial levers (28) are well extended outwards. The circular wheel (3) is designed to be at such a distance where it can be easily reached at by index finger of all possible hand sizes. In operative situations, the index finger can easily access any of these lateral levers (28) and can easily rotate the circular wheel (3). The easy and fluent rotation of this circular wheel (3) is made possible by allowing it to rotate on a frictionless rotary bearing (29).

Two lateral flanges (30) on the rotary bearing (29) engage into the corresponding grooves in the circular wheel (3). This way the rotation of the circular wheel (3) in turn rotates the bearing. The insulated elongate conduit (6) passes through this bearing and is fastened to it using two fastening screws (31), through two screw slots (32) from opposite direction. The rotation of the bearing effectively rotates the elongate conduit (6) which results into the corresponding movement of the working components (8).

The instrument is held in left or right hand. The index finger is placed in the trigger groove (15) beneath the zeppelin shaped oval belly (1), other fingers are inserted in the large fixed ring (4) and the thumb is placed in the small moveable ring (5). Using the opening and closing movement of hand, the handle is actuated. This motion is translated to the tip of the instrument through the rigid linkage and actuates the opening and closing of working components (8). The circular wheel (3) is rotated using index finger which rotates the whole elongate conduit (6) along with the working components (8) to 360 degrees. The ratchet mechanism becomes operational when the external locking slider (9) is pushed up where it gets temporarily locked. The ratchet mechanism is deactivated by pushing the external slider (9) down where it can be locked again.

1. An ergonomically designed laparoscopic instrument, comprising:
a trigger groove to receive an index finger;
a zeppelin shaped oval belly; and
a handle that includes a large fixed ring, a small moveable ring, a trigger groove to receive an index finger, and a circular wheel;
wherein the laparoscopic instrument provides an ergonomic grip.

2. The laparoscopic instrument of claim 1, further comprising:
working components to perform a surgical procedure;
wherein the ergonomic handle design enables a surgeon to use only one hand to manipulate the handle and to orient the working components into any desired operating angle, ratchet the working components while applying a desired clamping force and to actuate the working components as required to perform a surgical procedure.

3. The laparoscopic instrument of claim 1, further comprising:
a ratchet mechanism that includes an external locking slider positioned in the large fixed ring;
wherein the slider comprises an upward ratchet activation position and a downward ratchet deactivation position.

4. The laparoscopic instrument of claim 3, wherein the external locking slider includes a slider knob that is positionable between the middle and ring fingers of a user.

5. The laparoscopic instrument of claim 3, further comprising:
a ratchet arm between the large fixed ring and the small moveable ring, the ratchet arm operable to provide degrees of movement of the small moveable ring relative to the large fixed ring.

6. The laparoscopic instrument of claim 1, further comprising:
an elongate tube between the circular wheel and the working components;
wherein the circular wheel is operable to rotate the elongate tube.

7. The laparoscopic instrument of claim 6, wherein the circular wheel comprises four laterally projected radial levers placed at a distance from the zeppelin shaped oval belly such that the levers are accessable by the index finger for rotation thereby eliminating the need to use the other hand for rotating the elongate tube.

8. The laparoscopic instrument of claim 6, further comprising
a rotary bearing having two screw slots on opposing sides;
fastening screws with cone points that fasten with the rotary bearing to the elongate tube that passes through it, thereby allowing circular rotation of the elongate tube when the circular wheel and the rotary bearing rotate.

9. The laparoscopic instrument of claim 6, further comprising:
a high frequency (HF) cable connector assembly that includes
a based plate,
an HF cable connector mounted on the base plate, and
an angled conduit clamp attached to the HF cable connector, the angled conduit clamp having two curved plates which clasp around the elongate tube to provide definite electrical contact while allowing rotation of the elongate tube.

10. The laparoscopic instrument of claim 6, wherein the working components comprise:
a split tube attached to the elongate tube,
grasping forceps, dissecting forceps, tissue holding forceps and scissors and are pivoted by the split tube, and
a rigid linkage that runs through the length of the elongate tube and ends in a round ball positioned in a cavity in the upper part of the small moveable ring.

11. A laparoscopic instrument, comprising:
a housing with a rotateable knob;
a handle that extends from the housing and includes
a first fixed ring with a ratchet mechanism that includes an external locking slider positioned in the fixed ring wherein the slider comprises an upward lockable position and a downward lockable position
a second moveable ring,
a trigger groove to receive an index finger, and working components to perform a surgical procedure;
a rotateable elongate tube that attaches the housing to the working components, wherein the knob can be turned to rotate the elongate tube.

12. The laparoscopic instrument of claim 11, further comprising
a rotary bearing fastened to the elongate tube, thereby allowing circular rotation of the elongate tube when the circular wheel and the rotary bearing rotate.

13. The laparoscopic instrument of claim 11, further comprising:
a high frequency (HF) cable connector assembly that includes
a base plate,
an HF cable connector mounted on the base plate, and
an angled conduit claim attached to the HF cable connector, the angled conduit clamp having two curved plates which clasp around the elongate tube to provide electrical contact while allowing rotation of the elongate tube.

14. The laparoscopic instrument of claim 11, further comprising:
a rigid linkage that runs through the length of the elongate tube and ends in a round ball positioned in a cavity in the upper part of the second moveable ring.

15. A method of operating a laparoscopic instrument with one hand that includes, a handle with a first ring, a trigger groove, a second moveable ring, a circular wheel, a ratchet mechanism that includes an external locking slider positioned in the first ring, wherein the slider comprises an upward lockable ratchet activation position, a downward lockable ratchet deactivation position, working components to perform a surgical procedure and an elongate tube that attaches the handle to the working components, the method comprising:
grasping the laparoscopic instrument with one hand, with the index finger placed in the trigger groove, the middle and ring finger positioned on either side of the locking mechanism in the first ring, and the thumb placed in the second moveable ring;
opening and closing the working components by moving the moveable ring relative to the fixed ring;
rotating the circular wheel with fingers from the same hand that grasps the instrument to rotate the position of the working components; and
activation of the ratchet by pushing up the ratchet mechanism with the ring finger.

16. The method of claim 15, further comprising deactivation of the ratchet by pressing down on the ratchet mechanism with the middle finger.
